(19) United States
(12) Patent Application Publication
(10) Pub. No.: US 2010/0035231 A1
(43) Pub. Date: Feb. 11, 2010

(54) ANTIGEN CAPTURE ANTI-DENGUE IGA ELISA (ACA-ELISA) FOR THE DETECTION OF A FLAVIVIRUS SPECIFIC ANTIBODY

(75) Inventors: Bijon Kumarsil, Singapore (SG); Siew Lian Grace Yap, Singapore (SG)

Correspondence Address: KNOBBE MARTENS OLSON & BEAR LLP, 2040 MAIN STREET, FOURTEENTH FLOOR, IRVINE, CA, 92614, US

(73) Assignee: National Environment Agency, Singapore (SG)

(21) Appl. No.: 12/300,321

(22) PCT Filed: May 10, 2007
(86) PCT No.: PCT/SG2007/000132

§ 371 Date: Jun. 1, 2009

(30) Foreign Application Priority Data
May 11, 2006 (SG) 200603242-9

(51) Int. Cl. C12Q 1/70

(52) U.S. Cl. 435/5

(57) ABSTRACT

An antigen capture IgA Enzyme Linked Immunosorbent Assay (ACA-ELISA) was developed for the detection of anti-flavivirus IgA. The assay utilizes flavivirus lysate antigen, preferably dengue virus lysate antigen captured by a monoclonal antibody. Captured anti-flavivirus IgA from test sera are preferably detected using rabbit anti-IgA conjugated with a reporter group such as horseradish peroxidase (HRP). The assay was found to be at least 8 times more sensitive than anti-human IgA capture ELISA (AAC-ELISA). The ACA-ELISA, based either on serum or saliva, was found to be more sensitive and rapid compared to the “gold standard” anti-dengue IgM detection technique and can be utilized as a diagnostic tool for the confirmation of dengue in the early phase of infection.

### FIELD OF THE INVENTION

The present invention relates to a technique of detecting recent exposure of a human or an animal to a flavivirus or equivalent thereof. More particularly, the present invention relates to a rapid and easy analysis of a biological sample taken from a subject (animal or human) in order to determine if the subject had been exposed specifically to a flavivirus or equivalent thereof. The present invention further provides medical diagnostic kits and sero-evaluation by detecting flavivirus specific antibody (IgA) due to flavivirus infection or an equivalent thereof. Most preferably, the invention relates to the dengue virus.

### BACKGROUND

The Flaviviridae family contains a myriad of viruses that cause disease in humans and are generally transmitted by mosquitoes and ticks. The Flavivirus genus contains a number of viruses including yellow fever virus (YF), dengue fever (DF) virus, West-Nile (WN) virus and Japanese encephalitis (JE) virus which are responsible for their corresponding diseases.

Dengue is one of the major viral diseases affecting tropical and subtropical regions around the world, predominantly in urban and suburban areas. (DF) and its more serious forms, dengue hemorrhagic fever (DHF) and dengue shock syndrome (DSS), are important public health problems.

Dengue virus is a positive-stranded encapsulated RNA virus. The genomic RNA is approximately 11 kb in length and is composed of three structural protein genes that encode the nucleocapsid or core protein (C), a membrane-associated protein (M), an envelope protein (E), and seven nonstructural (NS) protein genes.

The gene order for dengue virus, as well as other flaviviruses, is 5′-C-prM(M)-E-NS1-NS2A-NS2B-NS3-NS4A-NS4B-NS5-3′. There are four distinct serotypes, serotypes 1 to 4. Infection induces a life-long protective immunity to the homologous serotype but confers only partial and transient protection against subsequent infections by the other three serotypes. Instead, it has generally been accepted that secondary infection or infection with secondary or multiple infections with various dengue virus serotypes is a major risk factor for DHF and DSS due to antibody-dependent enhancement. Other factors have been postulated to be important in the pathogenesis of DHF are; viral virulence, host genetic background, T-cell activation, the viral burden, and auto-antibodies. As attempts to eradicate Aedes aegypti, the most efficient mosquito vector of dengue virus, are not successful in countries where dengue is endemic, the control of dengue will be possible only after an efficient vaccine has been developed. At present, no effective dengue vaccine has been licensed.

The laboratory diagnosis of dengue virus infection can be made by the detection of specific virus, viral antigen, genomic sequence, and/or antibodies. At present, the three basic methods used by most laboratories for the diagnosis of dengue virus infection are viral isolation and characterization, detection of the genomic sequence by a nucleic acid amplification technology assay, and detection of dengue virus-specific antibodies. After the onset of illness, the virus is found in serum or plasma, circulating blood cells, and selected tissues, especially those of the immune system, for approximately 2 to 7 days, roughly corresponding to the period of fever.

Two patterns of serological response can be observed in patients with dengue virus infection: primary and secondary antibody responses, depending on the immunological status of the infected individuals. A primary antibody response is seen in individuals who are not immune to dengue or other member of flaviviruses. A secondary antibody response is seen in individuals who have had a previous dengue or flavivirus infection. For acute- and convalescent-phase sera, serological detection of antibodies based on capture immunoglobulin M (IgM) and IgG enzyme-linked immunosorbent assay (ELISA) has become the new standard for the detection and differentiation of primary and secondary dengue virus infections. This is important, since a sensitive and reliable assay for the detection and differentiation of primary versus secondary or multiple dengue virus infection is critical for the analysis of data for epidemiological, pathological, clinical, and immunological studies.

Progress toward the detection of antigen in acute-phase serum samples by serology has been slow due to the low sensitivity of the assay for patients with secondary infections, as such patients have pre-existing virus-IgG antibody immunocomplexes. However, recent studies that used ELISA and dot blot assays directed to the envelop membrane (E/M) antigen (the denKEY kit; Globio Co., Beverly, Mass.) and the NS1 antigen demonstrated that high concentrations of the E/M and NS1 antigens in the forms of an immune complex could be detected in the acute-phase sera of both patients with primary dengue virus infections and patients with secondary dengue virus infections up to 9 days after the onset of illness. Koraka et al. 2003. recently reported on the detection by a dot blot immunoassay of immune complex-dissociated NS1 antigen in patients with acute dengue virus infections and concluded that NS1 antigen detection by dot blot immunoassay in both non-dissociated and dissociated serum and plasma samples from patients with primary and secondary dengue virus infections results in the highest number of dengue antigen-positive patients compared with the numbers obtained by RT-PCR and with the denKEY kit.

The serological diagnosis of dengue virus infection is rather complicated for the following reasons: (i) patients may have multiple and sequential infections with the four dengue virus serotypes due to a lack of cross-protective neutralization antibodies; (ii) multiple and sequential flavivirus infections make differential diagnosis difficult due to the presence of pre-existing antibodies and original antigenic sin (many B-cell clones responding to the first flavivirus infection are restimulated to synthesize early antibody with a greater affinity for the first infecting virus than for the present infecting virus in every subsequent flavivirus infection) in regions where two or more flaviviruses are co-circulating; (iii) IgG antibodies have high degrees of cross-reactivity to homologous and heterologous flavivirus antigens; and (iv) the serodiagnosis of past, recent, and present dengue virus infections is difficult due to the long persistence of IgG antibodies (□10 months, as measured by E/M-specific capture IgG ELISA, or life long, as measured by E/M antigen-coated indirect IgG ELISA) in many dengue patients with secondary infections. Thus, among the viral infections that can be diagnosed by serology, dengue virus infection is most challenging.

Several methods have been described for the serological detection of dengue virus-specific antibodies, including the hemagglutination inhibition (HI) test, the neutralization test, the indirect immunofluorescent-antibody test, ELISA, complement fixation, dot blotting, Western blotting, and the rapid immunochromatography test. Among these, capture IgM and/or IgG ELISA, antigen-coated indirect IgM and/or IgG ELISA, and the HI test are the most commonly used serological techniques for the routine diagnosis of dengue virus infections. Traditionally, the HI test was used to detect and differentiate primary and secondary dengue virus infections due to its simplicity, sensitivity, and reproducibility. Patients are classified as having secondary dengue virus infections when the HI test titer in their sera is greater than or equal to 1:2,560 and are classified as having primary dengue virus infection if the HI test titer is less than 1:2,560. The HI test has recently become less popular and has gradually been replaced by the E/M-specific capture IgM and IgG ELISA due to the inherent disadvantages of the HI test.

Many rapid test kits that use the principle of immunochromatography are commercially available. Most of these kits can simultaneously detect IgM and IgG antibodies to dengue virus in human whole blood, serum, or plasma within 5 to 30 minutes. Some of these kits claim that it is possible to differentiate primary and secondary dengue virus infections, although this is not always reliable. The results showed that these kits generally have higher sensitivities for IgG detection but lower sensitivities for IgM detection and various specificities compared to the results of the E/M-specific capture IgM and IgG ELISA. Although the rapid test kits have the advantages of easy performance and rapid provision of results, they should at best serve as a screening test for clinicians in hospitals.

Some investigators have reported dengue virus-specific IgA and IgE antibody responses. (Talarmin et al., 1998.) reported on the use of an IgA- and IgM-specific capture ELISA for the diagnosis of dengue virus infection. The results showed that IgM appears more rapidly and lasts longer (between 2 to 3 months) than IgA (about 40 days). They concluded that the capture IgA ELISA is a simple method that can be performed together with the capture IgM ELISA and that can help in interpreting the serology of DF. More recently, Balmaseda et al., 2003 reported on the detection of specific IgM and IgA antibodies in serum and saliva. They concluded that dengue virus-specific IgA in serum has better potential to be a diagnostic target compared to saliva due to its higher level of performance.

The present invention provides an effective and sensitive detection method for the detection of IgA for a flavivirus infection which alleviates some of the problems of the prior art.

### SUMMARY OF THE INVENTION

The first aspect of the present invention provides a method for detecting IgA in a subject that is specific for a flavivirus or equivalent thereof said method comprising:
contacting a biological sample from the subject with a mixture of flavivirus specific immunogenic components; and
determining the presence of a complex that forms between a binding partner in the biological sample and a flavivirus specific immunogenic component; and
characterizing the binding partner in the complex with an anti-IgA antibody.

The method identifies only the IgA in the biological sample.

Another aspect of the present invention provides a method for detecting exposure of a subject to a flavivirus or equivalent thereof said method comprising:
contacting a biological sample from the subject with a mixture of flavivirus specific immunogenic components; and
determining the presence of a complex that forms between a binding partner in the biological sample and a flavivirus specific immunogenic component;
characterizing the binding partner in the complex; and
correlating the binding partner to exposure to the flavivirus.

The present invention results from a need to develop a rapid, low cost and straightforward assay to determine present or prior recent exposure to flavivirus. The invention preferably utilizes an antibody for the immuno-purification of flavivirus specific immunogenic components from cell lysate, which subsequently capture binding partners identified as anti-dengue IgA from a biological sample such as serum or saliva of flavivirus infected patients.

Accordingly, the present invention shows greater specificity and sensitivity compared to other conventional and most currently used dengue capture IgM ELISA by providing a platform that can specifically identify antibody (IgA) produced against flavivirus virus or immunological relatives thereof at an early stage of flavivirus infection. Most preferably, the method identifies dengue virus infection.

In another aspect of the present invention there is provided a solid support for use in a method for detecting exposure of a subject to a flavivirus or equivalent thereof, said method comprising:
contacting a biological sample from the subject with a mixture of flavivirus specific immunogenic components or an equivalent thereof;
determining the presence of a complex that forms between a binding partner present in the biological sample and a flavivirus specific immunogenic component; and optionally
characterizing the binding partner in the complex to correlate the binding partner to exposure to the flavivirus;
   said support comprising flavivirus specific immunogenic components immobilized on the support.

In a preferred embodiment, the biological sample may be applied to a polystyrene plate previously coated and captured with flavivirus antigen derived from flavivirus or an equivalent thereof added. Preferably the antigen or immunogenic component is derived from a cell lysate. The complex formed by an immunogenic component of the flavivirus cell lysate and the binding partner may then be detected using a detection agent that contains a reporter group and which specifically binds to the component/binding partner complex more specifically the IgA binding partners.

Yet another aspect of the present invention provides a kit for detecting IgA in a subject that is specific for a flavivirus or equivalent thereof or for detecting flavivirus exposure comprising:
a solid support including a flavivirus specific immunogenic component or equivalent thereof; or
a solid support including a flavivirus specific immunogenic component or equivalent thereof attached to a second support;
at least one detection agent conjugated to a reporter group for detecting a binding partner in a biological sample that forms a complex with the flavivirus specific immunogenic component; and optionally
instructions for using said kit to further identify the binding partner of the complex.

The present invention also provides individual components of the kit for use in the method of the present invention.

The present invention also serves as a method of assessing the relative risk of one or more subjects being exposed to flavivirus or an equivalent thereof within a defined location (e.g. geographical area, housing estate, means of transport or center for medical treatment or assessment), comprising;
obtaining samples from a representative population within a defined location; and
assessing evidence of exposure of individual members of a sample population to a flavivirus or equivalent thereof by the method comprising the steps of
   contacting a biological sample from the subject with a mixture of flavivirus specific immunogenic components; and
determining the presence of a complex that forms between a binding partner in the biological sample and a flavivirus specific immunogenic component and Wherein the presence of the complex is indicative of exposure of the subject to a flavivirus or equivalent thereof; and
assessing the relative risk of exposure within the defined location by characterizing the binding partner in the complex.

Risk analysis may be conducted using software in a computer readable form. Consequently, the present invention further relates to a computer readable program and computer comprising suitable for analysing exposure of subjects or group of subjects or a risk of exposure of subject or group of subjects to a flavivirus or equivalent thereof.

### FIGURES

FIG. 1 shows the optimization of dengue lysate antigen using the pan-dengue monoclonal antibodies coated polystyrene plate.

FIG. 2 shows the optimization of ACA-ELISA using dengue confirmed paired serum.

FIG. 3 shows the determination of cut-off point of ACA-ELISA using dengue confirmed negative and positive serum samples.

FIG. 4 shows the result of comparative sensitivities of two anti-dengue IgA ELISA techniques (ACA and MC).

FIG. 5 shows the comparative study of inhibition due to inhibition by non-dengue specific IgA in dengue-confirmed serum samples using 2 anti-dengue IgA ELISA techniques.

FIG. 6 shows the kinetics of anti-dengue IgA production using two ELISA techniques and its comparison with anti-dengue IgM using dengue confirmed-serum samples.

FIG. 7 shows the optimization of saliva-based ACA ELISA.

FIG. 8 shows the kinetics of anti-dengue IgA production in saliva detected by ACA-ELISA.

FIG. 9 shows the comparative performance of ACA-ELISA (saliva and serum) with dengue IgM ELISA (pan-bio) using dengue confirmed saliva samples.

### DETAILED DESCRIPTION OF THE INVENTION

The first aspect of the present invention provides a method for detecting IgA in a subject that is specific for a flavivirus or equivalent thereof said method comprising:
contacting a biological sample from the subject with a mixture of flavivirus specific immunogenic components; and
determining the presence of a complex that forms between a binding partner in the biological sample and a flavivirus specific immunogenic component; and
characterizing the binding partner in the complex with an anti-IgA antibody.

This method is specific for the identification of those binding partners in the biological sample that are IgA. This method may be further enhanced by the use of immunogenic components that have been isolated using a flavivirus specific IgA. Accordingly, the immunogenic components may be flavivirus IgA specific immunogenic components. The introduction of the IgA specific immunogenic components will attract the IgA in the biological sample which is specific for the flavivirus.

In another aspect of the present invention there is provided a method for detecting exposure of a subject to a dengue virus or equivalent thereof said method comprising:
contacting a biological sample from the subject with a mixture of flavivirus specific immunogenic components;
determining the presence of a complex that forms between a binding partner in the biological sample and a flavivirus specific immunogenic component;
characterizing the binding partner in the complex; and
correlating the binding partner to exposure to the flavivirus.

The present invention provides a new anti-dengue IgA detection technique (ACA-ELISA) which preferably targets saliva as an alternative to blood. Saliva contains high levels of IgA (19.9 mg/100 ml) compared to IgG and IgM (1.4 mg/100 mg and 0.2 mg/100 ml respectively). The technique shows a greater level of performance in terms of detecting anti-flavivirus IgA in saliva compared to serum and can be used as one of the early flavivirus diagnostics at the primary health care system where molecular-based flavivirus diagnostic is not available.

The present invention results from a need to develop a rapid, low cost and straightforward assay to determine present or prior recent exposure to flavivirus. In accordance with the present invention, the subjects including animals such as mammals and in particular humans are screened for the presence of binding partners preferably IgA to flavivirus or an equivalent thereof. The preferred binding partners are subject-derived binding partners such as, but not limited to immunointeractive molecules. The most immuno-interactive molecules are antibodies particularly immunoglobulin A (IgA). The identification of such binding partners is then used as evidence of present or prior recent exposure of the subject to flavivirus or an equivalent thereof.

The invention specifically utilizes an antibody, preferably a monoclonal antibody to capture antigen preferably from a flavivirus infected cell lysate, which includes a mixture of flavivirus immunogenic components including flavivirus particles amongst other antigens indicative of flavivirus virus infection. In the present invention the cell lysate preferably comprises a mixture of flavivirus immunogens, which includes virus particles and both the structural and non-structural viral proteins. Preferably the flavivirus immunogens are immunogenic components of the lysate that are capable of eliciting an immunological reaction to a binding partner in the biological sample.

Accordingly, the present invention shows greater sensitivity and specificity compared to other conventional and most currently used antibody capture IgA (AAC-ELISA) by providing a platform that can identify antibody produced against flavivirus or an equivalent thereof at an early stage of the infection.

Therefore, the present invention provides a new specific, rapid and economical detection method preferably using lysate of cells infected with flavivirus comprising a mixture of flavivirus components, preferably immunogenic components of the lysate described above, which permits the specific detection of flavivirus binding partners, preferably IgA that may be present in the test serum or in saliva. The test is rapid, preferably providing results within 90 minutes at room temperature (RT). Apart from it being a simple and convenient technique for specific antibody detection, one of the major advantages of the present invention is the preferred usage of flavivirus specific monoclonal antibody for the purification of flavivirus antigen from crude cell lysate and detection of anti-flavivirus IgA from saliva. Furthermore, the present invention makes the testing highly sensitive due to the maximum exposure of anti-dengue IgA present in human serum or saliva.

Throughout the description and claims of this specification, use of the word “comprise” and variations of the word, such as “comprising” and “comprises”, is not intended to exclude other additives, components, integers or steps.

Flaviviruses

The term “flaviviruses” or “flavivirus” as used herein the claims and description includes the Flaviviridae family of flaviviruses including the flavivirus genus which cause disease in humans and is generally transmitted by arthropods such as mosquitoes and ticks. The viruses are responsible for diseases such as but not limited to YF, DF and JE. The species of flaviviruses which make up the genus have demonstrated some conservation of sequences at the nucleotide and amino acid sequence level. The viruses included in the genus of flaviviruses include but are not limited to YF virus, dengue virus, WN virus and JE virus. Due to similarities at the nucleotide and amino acid level, these viruses may show similarities in antigenicity, transmission and disease. Most preferably, the flavivirus of the present invention is dengue virus.

Dengue Viruses

The term “dengue virus” is used herein the claims and description refers to all dengue serotypes (Den-1, Den-2, Den-3 and Den4) associated with a dengue infection. Preferably, the present invention is applicable to detecting dengue virus infection or exposure in any subjects including human, non-human animals and laboratory animals. Human subjects, however, are preferred in accordance with the present invention. However, the invention includes any subject that can respond to an infection or immunization by the dengue virus or an equivalent thereof.

A dengue virus is defined as a group of RNA human virus consisting of enveloped particles of about 40-50 nm in diameter. The viral genome is approximately 11 kb (Stollar et al, 1966). Mature virion consists of a positive sense RNA genome enclosed by an isometric nucleocapsid. The genome encodes a single open reading frame of about 11000 nucleotides, coding for the three structural (C-Capsid, M-Membrane and E-Envelope) and seven non-structural (NS1, NS2a and NS2b, NS3, NS4a and NS4b, NS5) proteins.

Dengue virus is transmitted to humans through the bites of infected female Aedes mosquitoes, principally the A. aegypti mosquito. This is a small, black and white, highly domesticated tropical mosquito that prefers to lay its eggs in artificial containers found in and around homes that may hold water such as buckets, flower vases and other water containers. The adult mosquitoes are rarely noticed outside; they usually rest in dark indoor sites, are unobtrusive and prefer to feed on humans or animals during the day light hours, with most biting activity occurring in the early morning or late afternoon (Gubler et al., 1992; Newton et al., 1992). The female mosquitoes are nervous feeders, disrupting the feeding process at the slightest movement of the host, thus returning to the same or different host to continue feeding. Because of this behaviour the mosquito often feeds on several persons during a single blood meal and if infective may transmit the virus to multiple persons (Platt et al, 1997; Scott et al., 1997). Such behaviour has been used to explain the epidermological observation that dengue diseases occur mainly in children although in certain places, like Singapore, this may have changed due to adaptation to vector control measures (Ooi et al., 2001).

Following the bite of an infective female mosquito, the virus undergoes an intrinsic incubation period of 3 to 14 days (average 4 to 7 days) after which the person may experience acute onset of fever accompanied by other non-specific signs and symptoms. During this viraemic period (which may be between 2 to 7 days) the virus circulates in the blood of infected humans. If an uninfected Aedes mosquito feeds on the host during this viraemic period, this mosquito will become infected after an obligatory extrinsic incubation period of 10 to 12 days. It would subsequently be able to transmit the virus to other uninfected hosts. In this transmission cycle, humans are the main amplifying hosts for the virus although studies show that monkeys can get infected and perhaps serve as a means of amplification for the virus (Putnam et al., 1995; Gubler et al., 1976; WHO, Fact sheets, 2002).

Dengue virus infection causes a spectrum of illness in humans depending on the infecting virus, the host's age and immunological conditions. It may result in asymptomatic illness or ranges from an undifferentiated flu-like illness (Viral syndrome) to DF, to DHF, and the severe and fatal DSS (Nimmannitya, 1993: WHO, 1997).

The World Health Organization has set up standards for the grading of the severity of DHF. There are four grades of severity of which grade III and grade IV are considered to be DSS.
Grade I: Fever with non-specific constitutional symptoms, and the only haemorrhagic manifestation is a positive tourniquet test and or easy bruising.
Grade II: In addition to the manifestation in grade 1, spontaneous bleeding in the forms of skin or other haemorrhages.
Grade III: Circulatory failure manifested by a rapid weak pulse, narrowing of pulse pressure or hypotension with the presence of cold, clammy skin and restlessness.
Grade IV: Profound shock with undetectable blood pressure or pulse (WHO, 1997).

Classical DF is more common in older children, adolescents and adults, and they are less likely to be asymptomatic (Sharp et al., 1995). The fever is abrupt in its onset with high fever, headache, incapacitating myalgias and arthralgias, nausea vomiting and macular or maculopapular rash (Waterman, 1989). The fever usually lasts for 5-7 days and sometimes can follow a biphasic course (Saddle back appearance) (Nimmannitya, 1993).

DHF is primarily a disease of the younger children below 15 years although it may also occur in adults and is mainly associated with secondary dengue infections (Sumarmo et al., 1983; WHO). The critical stage of DHF is at the time of defervescence, when the temperature becomes normal. The major factors that determine the severity of the illness at the time are plasma leakage due to the increased vascular permeability and abnormal homeostasis and other common hemorrhagic manifestations like petechiae, purpuric lesions, and ecchymoses. These symptoms plus a positive tourniquet test are helpful for accurate diagnosis of DHF (Gubler D J., 1998)

DSS is the terminal stage of DHF and is manifested by hypovolaemic shock due to plasma leakage (WHO, 1997). There are four warning signs of DSS: sustained abdominal pain, persistent vomiting, restlessness or lethargy, and a sudden change from fever to hypothermia with sweating and prostration. Early recognition and appropriate treatment by experienced hospital staff can decrease the case fatality rate of DSS to 0.2% but once shock sets in the mortality rate can be over 40% (Nimmannitya, 1994; Rigau-Perez J G, et al., 1998).

The E protein, the largest and the only structural protein exposed on the surface of the virus, is the major protein involved in immunological reactions such as receptor binding, haemmagglutination and neutralization. Infection in humans by one of the serotypes provides life long immunity to that serotype but only temporary protection against other serotypes.

The nucleoplasmid is in turn surrounded by lipid containing the envelope and membrane proteins. In addition to envelope and capsid proteins, dengue virus has seven non-structural proteins NS1, NS2a, NS2b, NS3, NS4a, NS4b and NS5.

Equivalents

The term “equivalent” as used herein and applied to the flavivirus virus is intended to include similar molecules that can elicit the same or similar response that the flavivirus or a structural or non-structural protein of the flavivirus could elicit. For instance, various antigens expressed by the flavivirus at various stages of infection or various virus particles or fragments may cause similar effects that the whole virus causes. The response may be an immunological response (non-clinical response) or it may be an infectious response (clinical response) or due to vaccination.

Exposure

The present invention is applicable to detecting exposure to the flavivirus or an equivalent thereof. Exposure may be present or prior exposure to the flavivirus or an equivalent thereof. Preferably, the exposure is sufficient to elicit an immune reaction or response in the body so as to induce a binding partner in response to the flavivirus or equivalent thereof. Once the subject is exposed, the method of the present invention may be applied at any stage of exposure as described above. Preferably, the method is used to detect exposure where there are no signs and symptoms that are obvious of a flavivirus infection. Preferably, the method detects exposure of the subject at a phase of flavivirus infection at an early acute phase for secondary infection or late convalescence stage of exposure to flavivirus or equivalent thereof for the primary infection or vaccination. The exposure may not always manifest in a flavivirus infection or notable signs or symptoms but it will cause a response so as to induce a binding partner. Preferably, the response is an immunological response.

The subject may have been exposed to flavivirus but need not show visual symptoms of the infection. The present method detects exposure that may lead to infection or may indicate prior exposure with no symptoms manifested.

Immune Response or Immunological Response

An “immune response” or “immunological response” is understood to be a selective response mounted by the immune system of vertebrates in which specific antibodies or fragments of antibodies and/or cytotoxic cells are produced against invading pathogens and antigens which are recognized as foreign in the body.

Binding Partner

The binding partner is any molecule or cell that is produced against the foreign dengue virus or equivalent thereof. Preferably, the binding partner is an antibody or immunologically active fragment thereof, or a cytotoxic cell. The binding partner includes an immuno-interactive molecule that can interact with a flavivirus antigen or equivalent and is preferably an IgA molecule.

As indicated herein, the preferred binding partner is an immuno-interactive molecule, which preferably refers to any molecule comprising an antigen binding portion or a derivative thereof. Preferably, the immuno-interactive molecule is an antibody against any portion of flavivirus proteins produced during a humoral response in the subject of a flavivirus virus infection or exposure.

As indicated herein the preferred binding partner is an antibody produced in the subject to a flavivirus or related virus components. However, a binding partner of the targeted antibody may also be used. An example of such a binding partner is an anti-idiotypic antibody or an antibody specific for and discriminatory of a subject antibody specific for a flavivirus or related virus components.

As used herein, an “anti-idiotypic antibody” is an antibody which binds to the specific antigen binding site of another antibody generated in response to exposure to a component derived from flavivirus or immunological relative thereof.

As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′)₂ fragments, and Fv fragments.

Preferably the binding partner is an antibody. More preferably it is a flavivirus IgA molecule or a dengue IgA molecule.

Biological Sample

The method of the present invention detects exposure to the flavivirus or equivalent thereof via the use of a biological sample obtained from a subject having been potentially exposed to the virus. The biological sample may be any sample from the body that may contain a binding partner. Such biological samples may be selected from the group including blood, saliva, cord fluid, B cells, T-cells, plasma, serum, urine and amniotic fluid. Preferably, the biological sample is serum or plasma. Most preferably, the biological sample is serum or saliva.

It is also preferred that the biological sample is obtained from subjects suspected of exposure to a flavivirus. A biological sample may also be modified prior to use, such as by dilution, purification of various fractions, centrifugation and the like. Accordingly, a biological sample may refer to a homogenate, lysate or extract prepared from a whole organism or a subset of its tissues, cells or component parts, or a fraction or portion thereof.

It should be noted that a biological sample might also be devoid of a binding partner that can interact with flavivirus or an equivalent thereof. This occurs when the subject has not been exposed to flavivirus or an equivalent thereof. Hence “determining the presence of a complex that forms between a binding partner in the biological sample and a flavivirus specific immunogenic component” may yield a zero result as a complex cannot form in the absence of binding partners. A control may be performed with flavivirus specific immunological agent such as a monoclonal antibody designed to compete with binding partners in the biological sample.

Reference to a biological sample being placed in contact with a component, preferably an immunogenic component or its immunological relative thereof should be understood as a reference to any method to facilitating the interaction of one or more immuno-interactive molecules of the biological sample with a component preferably of a cell lysate derived from infection of cells with the flavivirus or an equivalent thereof. The interaction should be such that coupling or binding or otherwise association between the immuno-interactive molecule and a specific immunogenic component of the lysate derived from cells infected with the flavivirus or an equivalent thereof can occur.

The biological sample is contacted with a mixture of flavivirus specific immunogenic components preferably derived from the lysate of cells infected with flavivirus or equivalent thereof. The lysate provides viral immunogenic components that may be provided by the flavivirus at any stage of its development. In the early convalescent stages of flavivirus infection, antibody preferably IgA derived from previous dengue infection is one of the indications of either secondary or primary flavivirus infection and this may be detected by the formation of a complex between it and a flavivirus specific immunogenic component, preferably an immunological component of the lysate.

Cell Lysate

The lysate as used in the present invention is preferably purified by immuno-purification using flavivirus specific monoclonal antibody. Importantly, the lysate is a mixture of components derived from a cell that has been infected by the flavivirus or equivalent thereof. The lysate is preferred source of the flavivirus specific immunogenic components. However, these components may be derived by other means. The cell lysate is the most convenient as the lysate can provide the earliest antigens produced by the virus and which can elicit an IgA response.

When a subject is exposed to flavivirus, the body reacts to initially remove the virus. This causes a chain of events generally manifesting in an immunological response to the plethora of antigens presented by the flavivirus or an equivalent thereof.

The lysate of the present invention may be obtained from any source of cells that have been infected with the flavivirus or equivalent thereof. Preferably, the cells are cells infected in an in vivo culture with flavivirus or equivalent thereof.

Any type of cell may be infected. Preferably, the cell type is capable of infection and culture of flavivirus. However, it is preferred that cells capable of producing high titres of flavivirus are infected in accordance with the methods of the present invention, including, but not limited to, continuous cell lines commonly available (e.g. Vero cells (Vero-PM strain), CV-1 cells, LLC-MK2, C6/36 and AP-61 cells), primary cell lines such as fetal Rhesus lung (FRhL-2) cells, BSC-1 cells, and MRC-5 cells, or human diploid fibroblasts. A combination of cell types is also envisaged by the present invention. C6/36 or AP-61 cells are infected with flavivirus or equivalent thereof. Most preferably the cell type is C6/36.

Cells may be cultured for any period, preferably for a period which allows the flavivirus to establish and infect the cell. More preferably, the cells are cultured until a cytopathic effect is apparent in the cell culture thereby indicating active infection of the virus in the cells.

At this point, the cells may be lysed by any method available to the skilled addressee. Generally, the use of a hypotonic buffer including a detergent such as Triton X may be used providing the lysing buffer does not affect the immunogens of the flavivirus or equivalent thereof but inactivates the live virus particles.

It should be appreciated that the lysate will contain a mixture of viral immunogenic components including structural and non-structural virus antigens as well as whole virus particles. For the dengue virus these may be selected from the group including DEN1, 2, 3 or 4. The present invention seeks to provide a mixture of antigens by a mixture of binding partners that are generated in the biological sample in response to exposure to the flavivirus or an equivalent thereof.

Preferably, the flavivirus is a dengue virus.

More preferably the flavivirus or dengue specific immunogenic component is a structural or non-structural protein of the flavivirus or dengue virus. More preferably, the structural protein is selected from the group including C-Capsid, M-Membrane and E-Envelope proteins, which may be captured by anti-flavivirus IgA. More preferably, the non-structural proteins for the dengue are selected from the group including NS1, NS2a, NS2b, NS3, NS4a, NS4b and NS5.

The lysate may be processed in any manner. Preferably, the lysate is clarified to remove nuclei and cellular debris and whole flavivirus particles. The lysate may be aliquoted and stored at −80° C. for future use.

For the dengue virus previous methods used specific dengue antigens DEN 1, 2, 3 and 4 (present in the supernatant of dengue virus infected cells) to detect antibodies indicative of dengue virus infection. However, the present invention does not solely use these antigens but a mixture of flavivirus molecules/immunogens (present in the flavivirus infected cells, which may contain flavivirus particles and other immunological components, preferably structural and non-structural proteins) against which antibodies develop in the course of flavivirus exposure.

The flavivirus specific immunogenic component is preferably from lysate and the biological sample is contacted so that a complex may form between the viral immunogenic components of the lysate and the binding partner contained within the biological sample. Preferably, immunogens of the flavivirus particles, including but not limited to those of the structural and non-structural proteins captured by anti-dengue IgA, will form complexes with binding partners. Preferably, the specific binding partners are antibodies or fragments thereof derived from the biological sample. These will only be present when the subject has been exposed/immunized to the dengue virus.

Formation of a Complex

A complex will form between an antibody, preferably an IgA for the dengue virus or equivalent thereof and a flavivirus specific/or reactive immunogenic component.

The methods and kits of the present invention seek to detect components and binding partners, which form complexes and are indicative of a flavivirus infection. These components and binding partners are generated in the course of a flavivirus infection.

The complex may comprise one or more binding partners bound to one or more components derived from flavivirus or an equivalent thereof. However, not all will be flavivirus specific IgA. Other molecules such as IgG and IgM may also bind.

The biological sample is left in contact with the component derived from flavivirus or an equivalent thereof for a period of time sufficient and conditions, which allow the stable formation of the complex or alternatively inhibits the attachment of a competitive immunological agent such as specific monoclonal antibodies (Mab).

The flavivirus specific immunogenic components and the biological sample are contacted so that a complex may form between the components and a binding partner present within the biological sample. Preferably, immunogens of the flavivirus particles, including but not limited to those of the structural and non-structural proteins preferably captured by anti-flavivirus IgA having an epitope specific to flavivirus, will form complexes with either binding partners or a competing flavivirus specific immunological agent such as a specific IgA. Preferably, the specific binding partners are antibodies or fragments thereof present in the biological sample. These will only be present when the subject has been exposed/immunized to the flavivirus.

Preferably, the complex will form between an antibody, preferably an IgA specific for the member of the flavivirus genus or equivalent thereof and a anti-flavivirus IgA captured flavivirus viral component. This is then indicative of flavivirus specific IgA in the sample and hence recent or prior exposure.

A competing flavivirus or member specific immunological agent will also form a complex with the component if the same epitope is remained free on the component. Where the binding partner and the immunological agent are specific for the same epitope, a competition will arise manifesting in an indication of the presence of the binding partner and prior exposure to flavivirus.

The preferred method of the present invention relies upon the detection of flavivirus specific binding partners preferably IgA present in the biological sample that are specific for a component of the flavivirus antigen present in cell lysate derived from a cell infected with the flavivirus or an equivalent thereof which has been captured using anti-flavivirus IgA. The complex may comprise one or more binding partners bound to one or more components derived from flavivirus or an equivalent thereof. However, it is the identification of an IgA bound to the complex which is indicative of prior exposure in this present invention.

Once attached, a competing flavivirus specific immunological agent may be added. Therefore, it is preferred to have a pre-incubation step where the binding agent and flavivirus specific immunogenic components are allowed to form a complex prior to the addition of the immunological agent. However, these components may also be added simultaneously.

Supports for the Detection of Flavivirus Specific IgA

Accordingly in another aspect of the present invention there is provided a solid support for use in a method for detecting exposure of a subject to a flavivirus or equivalent thereof, said method comprising:
contacting a biological sample from the subject with a mixture of flavivirus specific immunogenic components or an equivalent thereof;
determining the presence of a complex that forms between a binding partner present in the biological sample and a flavivirus specific immunogenic component; and optionally
the binding partner in the complex to correlate the binding partner to exposure to the flavivirus;
   said support comprising flavivirus specific immunogenic components immobilized on the support.

The solid support may be any material known to those of ordinary skill in the art to which a binding partner or flavivirus specific immunogenic component may be attached. For example, the solid support may be a test well in a microtitre plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The binding partner or the flavivirus specific immunogenic component may be immobilized on the solid support using a variety of techniques known to those of skill in the art, which are amply described in the patent and scientific literature. In the context of the present invention, the term “immobilization” refers to immuno-absorption or non-covalent association, such as adsorption, and covalent attachment (which may be a direct linkage between the antigen or nucleotide and functional groups on the support, or may be a linkage by way of a cross-linking agent). Immobilization by adsorption to a well in a microtitre plate or to a membrane is preferred. In such cases, adsorption may be achieved by contacting the binding partner or a component of the cell lysate, in a suitable buffer, with the solid support for a suitable amount of time. The contact time varies with temperature, but is typically about 1 hour and over night.

Covalent attachment of a binding partner or a flavivirus specific immunogenic component to a solid support may also generally be achieved by first reacting the support with a bifunctional reagent that will react with both the support and a functional group, such as a hydroxyl or amino group, of a binding partner or a component of the cell lysate. For example, the binding partner or component may be covalently attached to supports having an appropriate polymer coating using benzoquinone or by condensation of an aldehyde group on the support with an amine and active hydrogen of the component (see, for example, Pierce Immunotechnology Catalog and Handbook, 1991, at A12-A13).

Detecting, Characterizing and Identifying the Binding Partner of the Complex

The flavivirus specific immunogenic component derived from a flavivirus or an equivalent thereof is then left in contact with the biological sample for a period of time sufficient and under conditions, which allow the stable formation of a complex. Once a complex is formed, a detection system is then added to facilitate the detection of the specific binding of the binding partner in the complex to the flavivirus specific immunogenic components.

Detecting the complex between the components derived from flavivirus or an equivalent thereof and a subject-derived binding partner such as immunoreactive molecules, may be based on any convenient method, which will be known to those of the skill of the art.

It is contemplated that procedures useful for detecting components and binding partners which form complexes and are indicative of a flavivirus infection in a biological sample include, but are not limited to, immunological assays, such as immunoblotting, immunocytochemistry, immunohistochemistry or antibody-affinity chromatography, Western blot analysis, or variations or combinations of these or other techniques such as are known in the art.

In general, components and binding partners, which form complexes and are indicative of a flavivirus infection, may be detected in a biological sample obtained from a subject by any means available to the skilled addressee. In a preferred embodiment, the method of detection employs a further detection agent such as specific MAb and anti-MAb conjugated with enzyme, which permits detection of said complexes and the binding partners.

In a preferred embodiment, the methods as herein described involve the use of a cell lysate derived from a cell infected with a flavivirus or an equivalent thereof or the purified components there from (herein referred to interchangeably as the “components” or the “components of the cell lysate”), immobilized on a solid support such as a polystyrene or nitrocellulose membrane to which a binding partner of a biological sample may absorb/bind. The complex formed by a component of the cell lysate and the binding partner may then be detected using a detection agent that contains a reporter group and specifically binds to the component/binding partner complex. Such detection agent may comprise, for example, an antibody or other agent that specifically binds to the binding partner, such as an anti-immunoglobulin (i.e. antibody), protein G, protein A or a lectin. Alternatively, a competitive assay may be utilized, in which a detection agent capable of binding an antigen derived from a flavivirus is labeled with a reporter group and allowed to bind to the immobilized component of the cell lysate in combination with the binding partner of the biological sample. The extent to which the binding partner of the biological sample inhibits the binding of the labeled flavivirus detection agent to the immobilized component is indicative of the reactivity of the binding partner of the biological sample with the immobilized component.

In a preferred embodiment, the detection reagent is an antibody or secondary antibody or an antigen-binding fragment thereof, capable of binding to the binding partner of the biological sample. Antibodies may be prepared by any of a variety of techniques known to those of ordinary skill in the art (See, for example, Harlow and Lane, Antibodies: A Laboratory Manual, Cold Spring Harbor Laboratory, 1988). In general, antibodies can be produced by cell culture techniques, including the generation of MAb, or via transfection of antibody genes into suitable bacterial or mammalian cell hosts, in order to allow for the production of recombinant antibodies.

The secondary antibody which may be conjugated to a label, can be added to the complex to facilitate detection. A range of labels providing a detectable signal may be employed. The label may be selected from a group including chromogen, an enzyme, a catalyst, a fluorophore and a direct visual label. In the case of a direct visual label, use may be made of a colloidal metallic or non-metalic particle, a dye particle, an enzyme or a substrate, an organic polymer, or a latex particle. A large number of enzymes suitable for use as labels are disclosed in U.S. Pat. Nos. 4,366,241, 4,843,000 and 4,849,338. Suitable enzyme labels in the present invention include alkaline phosphates, horseradish peroxidase, preferably horseradish peroxidase. The enzyme label may be used alone or in combination with a second enzyme, which is in solution. In the present invention a secondary antibody attached with horseradish peroxidase, which then reacts with its substrate DAB and produces a visually detectable colour change, preferably achieves the detection of the complex.

Preferably, the antibody is an anti IgA antibody and therefore detects IgA binding partners that have bound to the flavivirus specific immunogenic components.

General Description of the Process

This assay may be performed by first contacting a binding partner of a biological sample that has been immobilized on a solid support, commonly the well of a microtitre plate, with the flavivirus specific immunogenic components as herein described, such that a component is allowed to bind to the immobilized binding partner such as an antibody. Alternatively, the flavivirus specific immunogenic components may be bound to the solid support such that binding partners are allowed to bind to the immobilized component. Unbound sample is then removed from the immobilized complex and a detection reagent (preferably a second antibody capable of binding to the binding partner or the component, containing a reporter group) is added. The amount of detection reagent that remains bound to the solid support is then determined using a method appropriate for the specific reporter group.

More specifically, once the binding partner or a flavivirus specific immunogenic component is immobilized on the support as described above, the remaining binding sites on the support are typically blocked. Any suitable blocking agent known to those of ordinary skill in the art, such as bovine serum albumin or skin milk with either Triton X 100 or Tween 20™ (Sigma Chemical Co., St. Louis, Mo.). The component or binding partner may also be diluted with a suitable diluent buffer, such as phosphate-buffered saline (PBS) with human serum and either Triton X 100 or Tween 20 prior to incubation. In general, an appropriate contact time (i.e., incubation time) is a period of time preferably 30 minutes that is sufficient to allow a flavivirus specific immunogenic component to bind to the immobilized binding partner, or vice versa. Preferably, the contact time is sufficient to achieve a level of binding to the target epitope on the attached flavivirus specific immunogenic component that is at least about 95% of that achieved at equilibrium between the bound and unbound binding partner or flavivirus specific immunogenic component. Those of ordinary skill in the art will recognize that the time necessary to achieve equilibrium may be readily determined by assaying the level of binding that occurs over a period of time. At room temperature (RT), an incubation time of about 30-60 minutes is generally sufficient.

Unbound flavivirus specific immunogenic component or binding partners may then be removed by washing the solid support with an appropriate buffer, such as PBS containing 0.05% Tween 20™ or Tween 80. A detection agent which is capable of binding to the binding partner, and which contains a reporter group, may then be added. The detection agent is generally an anti-IgA antibody. Preferred reporter groups include those groups recited herein. The detection agent is then incubated with the immobilized binding partner-component complex for an amount of time sufficient to detect the bound component or binding partner. An appropriate amount of time may generally be determined by assaying the level of binding that occurs over a period of time. Unbound detection agent is then removed and bound detection agent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups, chromogenic enzymes and fluorescent groups. Chromogenic enzymes include, but are not limited to, peroxidase and alkaline phosphatase. Fluorescent groups include, but are not limited to, fluorescein isothiocyanate (FITC), tetramethylrhodamine isothiocyanate (TRITC), rhodamine, Texas Red and phycoerythrin. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products. The substrate can be selected from a group of agents consisting of 4-chloro-l-naphtol (4CN), diaminobenzidine (DAB), aminoethyl carbazole (AEC), 2,2′azino-bis(3-ethylbenzothiazoline-6-sulfonic acid) (ABTS), ophenylenediamine (OPD) and tetramethyl benzidine (TMB).

It may also be desirable to couple more than one reporter group to a detection agent. In one embodiment, multiple reporter groups are coupled to one detection agent molecule In another embodiment, more than one type of reporter group may be coupled to one detection agent. Regardless of the particular embodiment, detection agents with more than one reporter group may be prepared in a variety of ways. For example, more than one reporter group may be coupled directly to a detection agent, or linkers that provide multiple sites for attachment can be used.

In a related embodiment, the method as herein described may be performed in a flow-through or strip test format, wherein the binding partner of a biological sample or a flavivirus specific immunogenic component is immobilized on a membrane, such as nitrocellulose. In the flow-through test, for example, a flavivirus specific immunogenic component is capable of binding to the immobilized binding partner as the sample passes through the membrane. Alternatively, a binding partner in a biological sample is capable of binding to the immobilized flavivirus specific immunogenic component as the sample passes through the membrane. A second, labeled detection agent then binds to the binding partner-component complex as a solution containing the detection agent flows through the membrane. The detection of bound detection agent may then be performed as described above.

In the strip test format, one end of the membrane to which a component of the cell lysate is bound is immersed in a solution containing the biological sample. The binding partner in the biological sample migrates along the membrane through a region containing a detection agent and to the area of the immobilized component. The concentration of detection agent at the area of immobilized binding partner-component complex indicates the presence of binding agent in a biological sample. Typically, the concentration of the detection reagent at that site generates a pattern, such as a line, that can be read visually. The absence of such a pattern indicates a negative result. In general, the amount of binding partner immobilized either on the membrane or polystyrene plate is selected to generate a visually discernible pattern when the biological sample contains a level of a binding agent that would be sufficient to generate a positive signal in the sandwich assay, in the format discussed above. Such tests can typically be performed with a very small amount of biological sample.

In a simpler version of the strip test or dipstick test, components of the cell lysate can be immobilised onto a membrane, such as a nitrocellulose membrane. Strips of membrane may then be subjected to biological samples to form complexes between the components and a binding partner in the biological sample. The complex is then detectable by any means described above using a detection agent, such as an antibody. The dipstick test can provide a quick indication of previous exposure without using large biological samples.

As used herein, “binding” refers to a non-covalent association between two separate molecules such that a complex is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant may be determined using methods well known in the art.

Membranes contemplated by the method and kits of the present invention include any membrane to which either the binding partner or components derived from the flavivirus or equivalent thereof can bind. Examples of membranes include without being limited to, nitrocellulose membranes, polytetrafluorethylene membrane filters, cellulose acetate membrane filters and cellulose nitrate membrane filters with filter paper carriers. Most preferably, the membrane is a nitrocellulose membrane.

Alternatively, the diagnostic methods of the present invention may adopt an automated analytic method using a biological microchip. For instance, a diagnostic kit can be structured to perform immuno-blotting using a glass slide coated with the component of the cell lysate. This diagnostic kit may comprise a biological microchip onto the surface of which a flavivirus specific immunogenic component is immobilized, an appropriate buffer, a standardised sample comprising a detectable level of binding agent, and a secondary detection reagent, as herein described.

The method and kits of the present invention can detect specific exposure of human or animals to a flavivirus or any specific member of the family or equivalent thereof either during acute infection or in convalescent phase. As used therein “acute infection” refers to the period of time when a virus has infected a host and is actively replicating and/or causing symptoms associated with infection like fever, rush, joint pain and or abdominal pain. The “convalescent phase” refers to the stage of flavivirus infection cycle when flavivirus virus is no longer multiplying or remains in the host blood and has developed binding partners such as, but not limited to antibodies. Using the method and kit of the present invention, exposure can be detected at any time after generation of a binding partner in the infected patient or patient derived it from his/her previous infection/infections.

Kits

In another aspect of the present invention there is provided a kit for detecting IgA in a subject that is specific for a flavivirus or equivalent thereof or for detecting flavivirus exposure comprising:
a solid support including a flavivirus specific immunogenic component or equivalent thereof; or
a solid support including a flavivirus specific immunogenic component or equivalent thereof attached to a second support;
at least one detection agent conjugated to a reporter group for detecting a binding partner in a biological sample that forms a complex with the flavivirus specific immunogenic component; and optionally
instructions for using said kit to further identify the binding partner of the complex.

Optionally, the kit will also include additional parts such as washing buffers, incubation containers, blocking buffers and instructions as are necessary for conducting the method.

Accordingly the present invention provides a kit for detecting exposure of a subject to flavivirus or any member of the family or an equivalent thereof. The kit may be any convenient form which allows for a binding partner in a biological sample to interact with an anti-dengue IgA captured flavivirus viral component and may further compete with a competing flavivirus specific immunological agent. The result is an indication, by the presence of flavivirus specific flavivirus specific-binding partners such as IgA in the biological sample, of prior exposure to flavivirus. Preferably the kit comprises a solid support such as described herein adapted to receive or comprise anti-flavivirus IgA captured components of flavivirus or an equivalent thereof. The kit may also comprise reagents, reporter molecules capable of providing detectable signals and optionally instructions for use. The kit may be in modular form wherein individual components may be separately purchased.

The kit may be a modular kit comprising one or more members wherein at least one member is a solid support comprising an anti-flavivirus IgA captured flavivirus component of flavivirus or equivalent or cell lysate comprising an immunogenic component derived from a flavivirus or equivalent thereof.

In an alternative embodiment the solid support comprises an array of binding partners for one or more components of one or more flavivirus or equivalent thereof from one or more subjects.

The present invention also provides individual components of the kit for use in the method of the present invention. The invention provides solid supports which include anti-flavivirus IgA captured components of the flavivirus for use in the detection of exposure to the flavivirus. In one embodiment, the invention provides a polystyrene 96 well plate or a nitrocellulose membrane to attach viral antigen, either for use as an immobilized anti-flavivirus IgA captured flavivirus viral components or as a dot blot or use as a dip stick, which includes components of the flavivirus or equivalent thereof. Preferably, the plate or membranes include components selected from the group including flavivirus structural and non-structural proteins, flavivirus particles and fragments thereof, glycoproteins, lipids and carbohydrates derived from the flavivirus or any mixture thereof.

The solid support may also be a microtitre plate, glass slide or biological microchip wherein the components of the cell lysate are immobilised. These solid supports can then be subjected to the biological sample to detect flavivirus exposure. Preferably polystyrene microtitre plate is used to attach flavivirus antigen by immuno-purification using anti-flavivirus IgA from flavivirus infected cell lysate.

In the case of a nitrocellulose membrane, the second support may be a holder, which holds the solid support to improve manipulation of the solid support, which has immobilised components of the flavivirus. For instance, the nitrocellulose membrane may be supported on a stick that enables the membrane to be dipped into a biological sample such as serum. This is useful as a component of a kit since small amounts of biological sample can be tested simultaneously.

Assessing Relative Risk of Infection

In yet another aspect, the present invention also provides a method of assessing the relative risk of one or more subjects being exposed to flavivirus or an equivalent thereof within a defined location (e.g. geographical area, housing estate, means of transport or center for medical treatment or assessment), comprising;
obtaining samples from a representative population within a defined location; and
assessing evidence of exposure of individual members of a sample population to a flavivirus or equivalent thereof by the method comprising the steps of
   contacting a biological sample from the subject with a mixture of flavivirus specific immunogenic components; and
   determining the presence of a complex that forms between a binding partner in the biological sample and a flavivirus specific immunogenic component and wherein the presence of the complex is indicative of exposure of the subject to a flavivirus or equivalent thereof; and
   assessing the relative risk of exposure within the defined location by characterizing the binding partner in the complex.

Risk analysis may be conducted using software in a computer readable form. Consequently, the present invention further relates to a computer readable program and computer comprising suitable for analysing exposure of subjects or group of subjects or a risk of exposure of subject or group of subjects to a flavivirus or equivalent thereof.

The method or technique of the present invention allows for the epidemiological study or sero-surveillance of outbreaks of infection caused by flavivirus or any member of the family or equivalent thereof. Such studies provide valuable information, which advance multiple facets of research in the area of flavivirus disease. For example, epidemiological studies aid in the identification of the index of an infection. Such information enables the identification of a defined location from which the source of virus responsible for a viral outbreak originated.

Additionally the technique/method of the present invention permits for the rapid identification or isolation of subjects who are infected with a flavivirus or equivalent thereof without major laboratory equipment or even in field conditions. Such information aids in identifying subjects, who require medical treatment as well as defining locations that require further investigation or disease control approaches such as identification of breeding places and its control. Further, the technique of the present invention allows for the monitoring of an infected patient to determine the presence of—anti-flavivirus specific IgA. Alleviation of IgA titre or its presence in an early phase of infection may be the indication of secondary infection and thus, help the monitoring of the subsequent phases of flavivirus infection like DHF or DSS.

Further, the technique of the present invention provides a means for identifying subjects who are infected with any specific member of the genus of flavivirus and serotypes involved, allowing for the rapid detection, risk of further infection, pointing to the location of an infection and disease control strategy.

A reference herein to a patent document or other matter which is given as prior art is not to be taken as an admission that the document or matter was known or that the information it contains was part of the common general knowledge as at the priority date of any of the claims.

Examples of the procedures used in the present invention will now be more fully described. It should be understood, however, that the following description is illustrative only and should not be taken in any way as a restriction on the generality of the invention described above.

### EXAMPLES

### Example 1

### Development of Anti-Dengue IgA (ACA-ELISA) Using Serum

a) Preparation of antigen: Lysate dengue viral antigens were prepared against all four serotypes of dengue virus according to the method described by Cardosa et al., 2002. Briefly, dengue viruses (5 m.o.i.) were grown in C6/36 cells with virus maintenance medium containing 2% fetal calf serum were incubated 4-5 days depending on the development of cytopathic effects and serotypes of the virus. The medium was decanted and the flask with infected cells was washed four times with PBS, treated with 1 ml hypotonic buffer with 1% trix100 for an hour and finally centrifuged at 14000 rpm for 10 minutes. The supernatant was collected, allocated 250 μl in eppendrofs and stored at −70° C. until use.

b) Serum samples: A total 292 dengue PCR confirmed serum samples in 3 sets were used as positive samples. 182 patient serum samples negative to dengue PCR were used as negative samples in this study.

c) Serological assays: IgM capture ELISA, a commercial kit (Pan-bio, Australia) was used for the measurement of anti-dengue specific IgM antibodies in the samples used in this study. The test was performed according to the procedures described by the manufacturer.

IgG indirect ELISA, a commercial kit (Pan-bio, Australia) was used for the detection of anti-dengue specific IgG antibodies in serum samples used in this study. The test was performed according to the procedures described by the manufacturer.

d) IgA capture ELISA (AAC-ELISA): The method described by Talarmin et al., 1998 and Balmaseda et. al, 2003 was used with minor modification. Briefly a 96 well polystyrene plate (maxi-absorb, NUNC) was coated with 100 μl of anti-human IgA (Source) diluted at 1:500 in coating buffer (sodium bicarbonate buffer, Sigma, USA) and incubated either 2 hours at 37° C. or overnight at 4° C. Wells were blocked with blocking buffer (5% skim milk containing 0.1% triton X100) for an hour at 37° C. and washed 4 times with washing buffer (1× PBS containing 0.05% Tween 20). 10 pairs of dengue confirmed serum samples (anti-dengue IgM and IgG) were used to optimize the test. Each serum sample was diluted at 1:100 in diluent buffer (5% skim milk containing 0.1% triton X100) and 100 μl of diluted serum sample was added to each well and incubated for 1 hour at RT. 1 positive and 3 negative control wells were kept in each plate. A mixture of dengue lysate antigen (1:100) and pan dengue reactive monoclonal antibody conjugated with HRP (1:1000) (ICL, USA) was prepared in a glass bottle and incubated at RT for an hour. Following six (6) washes of the plate as described above, 100 μl of antigen and MAb mixture was added to each well and incubated for 1 hour at RT and then washed again 6 times. 100 μl of OPD (Sigma. UK) was then added to each well and incubated at RT for 5-10 minutes. The reaction was then stopped with stopping buffer (2.75% sulphuric acid) and the plate read in an ELISA reader at 492 nm. Results were calculated using a formula of OD value of the test sample divided by an average OD of negative samples and multiplying the value by 5.

e) Antigen captures anti-dengue IgA ELISA (ACA-ELISA): The 96 well plate (Max-absorb-NUNC) was coated with 100 μl per well of anti-mouse IgG diluted at 1:1000 in coating buffer and incubated either over-night at 4° C. or 1 hour at 37° C. After blocking the plate with blocking buffer for 1 hour at 37° C., 100 μl of pan-dengue MAb (ICL, USA) diluted at 1:1000 in PBS was added to each well and incubated at 37° C. for an hour. After 4 washes, dengue lysate antigen (1-4) was added at 1:100 dilutions to each well and incubated again at 37° C. for an hour. The plate was then washed 4 times using washing buffer and 100μl of test serum at 1:100 in diluent buffer was added to each well. 1 positive and 3 negative serum controls were kept in each plate. After 1 hour of incubation at RT, the plate was washed again 6 times using wash buffer and 100 μl of rabbit anti-human IgA conjugated with HRP (1:4000) was added to each well and further incubated for 30 minutes at RT. Following incubation, the plate was washed again 6 times and 100 μl of OPD (Sigma, USA) was added to each well and incubated for 5 minutes at RT. Further colour development was stopped using sulphuric acid (2.75%) and the plate was read in an ELISA reader at 492 nm. Results were calculated using a formula of OD value of the test sample divided by an average OD of negative samples and multiplying the value by 5.

f) Comparative analytical sensitivity of ACA and AAC ELISAs The analytical sensitivity of IgA captured (AAC) and antigen captured (ACA) enzyme assays for the detection of anti-dengue IgA in patient's serum was studied by using strong, medium and weak dengue IgA positive serum samples. Serum samples were diluted at 1:5 to 1:640 either in serum from a healthy person who is negative to dengue antibodies (IgG, IgM and IgA) or in diluent buffer and used as stock solutions. The stock diluents were further diluted at 1:100 in diluent buffer as working solutions and the assays (MC and ACA ELISA) were performed as described above. The inhibitions of the assays due to dengue non-specific IgA in serum against diluent buffer were calculated by the formula given below:

Percentage of inhibition (PI)=100−(OD values in serum diluent/OD values in diluent buffer)×100.

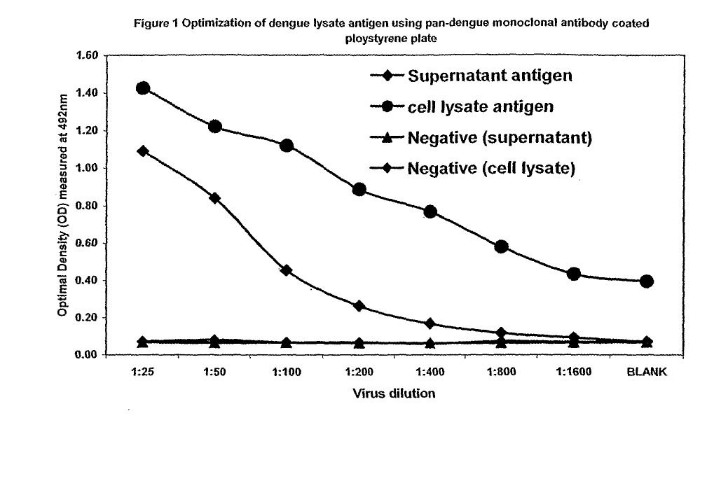

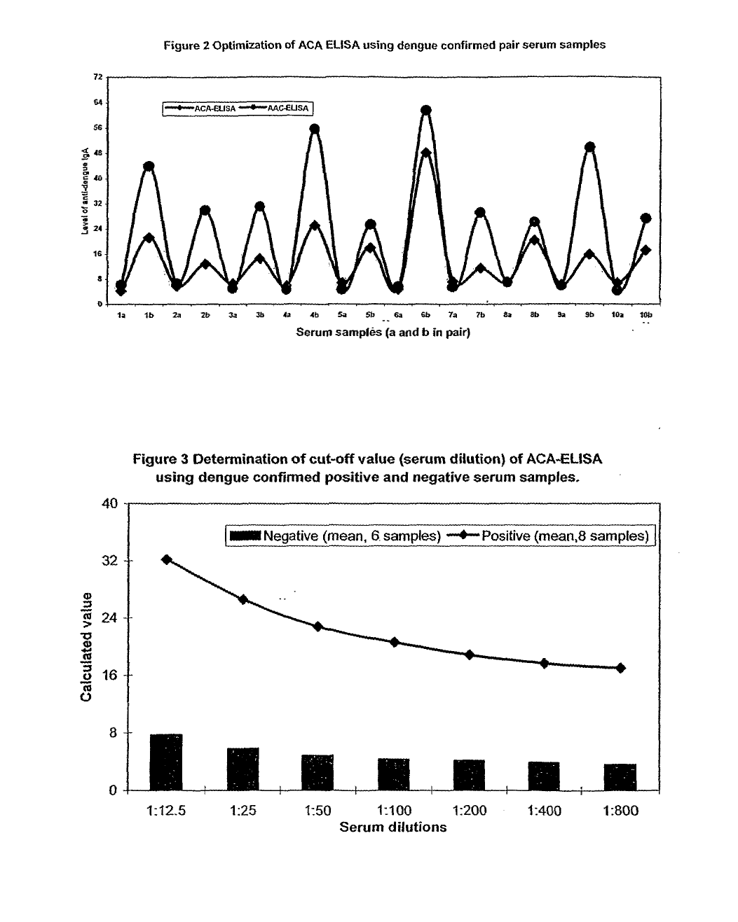

g) Standardization of assays: Checkerboard titrations showed that 100 μl of anti-mouse IgG, 100 μl pan dengue MAb (1:1000 in PBS, and 100 μl of lysate antigen at 1:100 dilution) was found as optimum for the assay in 96 well plates (FIG. 1). The optimal dilution of serum samples (1:100) used in the ELISA was determined by checkerboard titration against anti-human IgA using 10 dengue positive and 6 dengue negative antibody serum samples (FIG. 2). Similarly, the optimal dilutions of rabbit anti-human IgA-HRP for ACA-ELISA was determined by checkerboard titration and was optimized at 1:4000.

The serum dilution of the assay was carried out using a serial dilution of samples ranging from 12.5 to 1:800. 8 anti-dengue IgA positive and 6 anti-dengue IgA negative serum samples were used in this assay. A mean of all positive serum samples were found positive even at 1:800 dilution while none of the negative samples was positive (FIG. 3). 2 samples were slightly above cut-off point and serum dilution for the assay was fixed 1:100 (4 times negative samples dilution) and used through the assay.

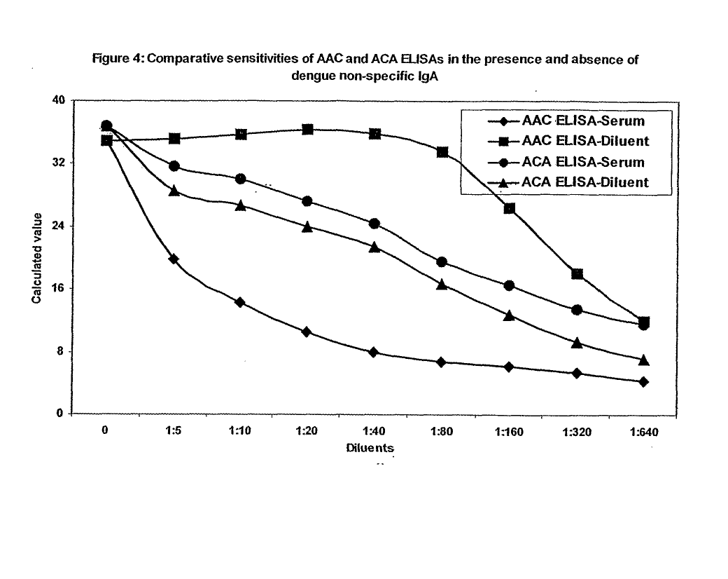

The ACA-ELISA was developed using 10 pairs of dengue PCR positive acute and convalescent serum samples collected at 10-37 day interval. Sera were tested at 1:100 and results showed that all dengue confirmed 10 convalescent sera showed high levels of anti-dengue IgA against dengue lysate antigen (FIG. 4).

### Example 3

### Comparative Analytical Sensitivity of Two IgA Assays:

The level of sensitivities of two anti-dengue IgA assays (MC-ELISA and ACA-ELISA) was further analyzed using dengue positive IgA serum samples in 2 different diluents such as dengue negative serum and diluent buffer. Results in FIG. 4 showed that in AAC-ELISA, serum having a high level of anti-dengue IgA, when diluted in negative serum, was found 32 times less sensitive than in diluent buffer and the inhibition due to high levels of non-dengue specific IgA in serum diluent varying from 43.71% to 79.79 % (FIG. 4).

On the other hand, in ACA-ELISA the level of detections of dengue specific lgA were equal in both the diluents (negative serum and diluent buffer) and the inhibition of non-dengue specific IgA present in serum diluent was negligible, varying from −11.08% to −61.76% (FIG. 4).

### Example 4

### The Sensitivity and Specificity of ACA-ELISA

This was carried out using 296 dengue confirmed and 182 dengue negative serum samples collected at acute and convalescent stages. Out of the dengue confirmed samples, 96 were collected between days 1-3 of onset of fever, 97 samples between 3-7 days while 102 were collected between days 10-37 of onset of fever. 182 serum samples were collected from patients who were negative to dengue PCR and had developed fever during the collection of samples. The sensitivity and specificity of ACA- and AAC-ELISAs are showed in Tables 1 and 2.

TABLE 1
Sensitivity and specificity of serum ACA-ELISA

| | | Dengue real-time PCR | | |
|---|---|---|---|---|
| | | Positive | Negative | Total |
| ACA-ELISA | Positive | 180 | 16 | 196 |
| | Negative | 112 | 166 | 278 |
| | Total | 292 | 182 | 474 |
| Sensitivity (%) | 61.64 | Specificity (%) | 91.21 | |
| PPV (%) | 91.84 | NPV (%) | 59.71 | |

TABLE 2
Showing the sensitivity and specificity of AAC-ELISA

| | | Dengue real-time PCR | | |
|---|---|---|---|---|
| | | Positive | Negative | Total |
| AAC-ELISA | Positive | 95 | 3 | 98 |
| | Negative | 138 | 179 | 317 |
| | Total | 233 | 182 | 415 |
| Sensitivity (%) | 40.77 | Specificity (%) | 98.35 | |
| PPV (%) | 96.94 | NPV (%) | 56.47 | |

### Example 5

### Kinetics of Anti-Dengue IgA and IgM Production Among Dengue Confirmed Serum Samples Using Two Anti-Dengue IgA Assays and IgM Antibody Capture (MAC)-ELISA

The kinetics of IgA (MC and ACA-ELISAs) and IgM was carried out using 101 dengue confirmed serum samples in 3 sets. A total of 292 samples were collected at acute and convalescent stages. Each serum samples was collected in 3 collections {first collection (1-3 days), second collection (3-7 days) and third collection (10-37 days)} after the onset of fever. Out of the dengue positive serum samples, 35.79% were positive to anti-dengue IgA on the first collection (1-3 days), 61.46% were positive on the second collection (3-7 days) while 85.15% serum samples were positive on the third collection (10-37 days).

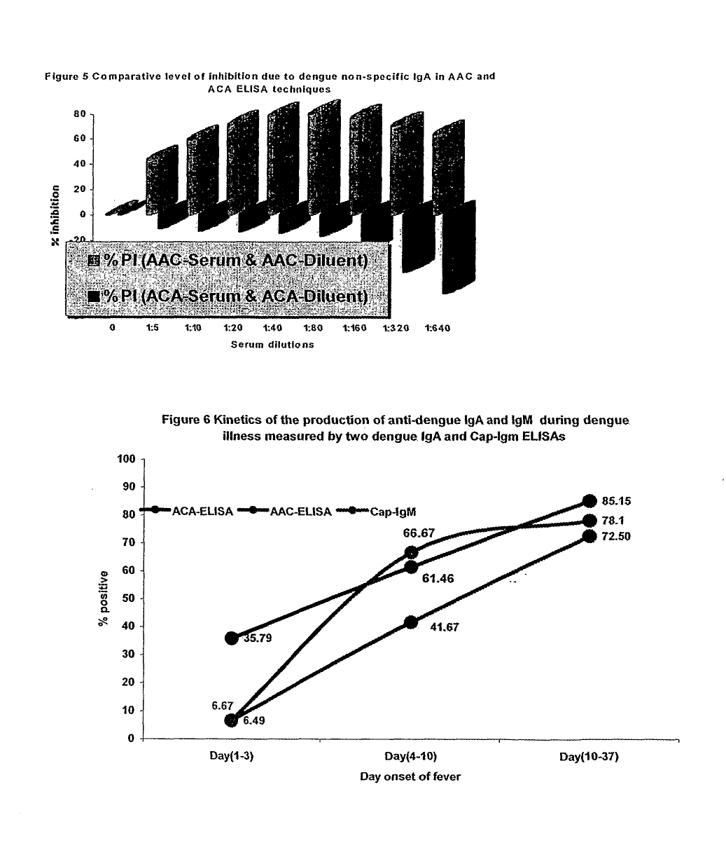

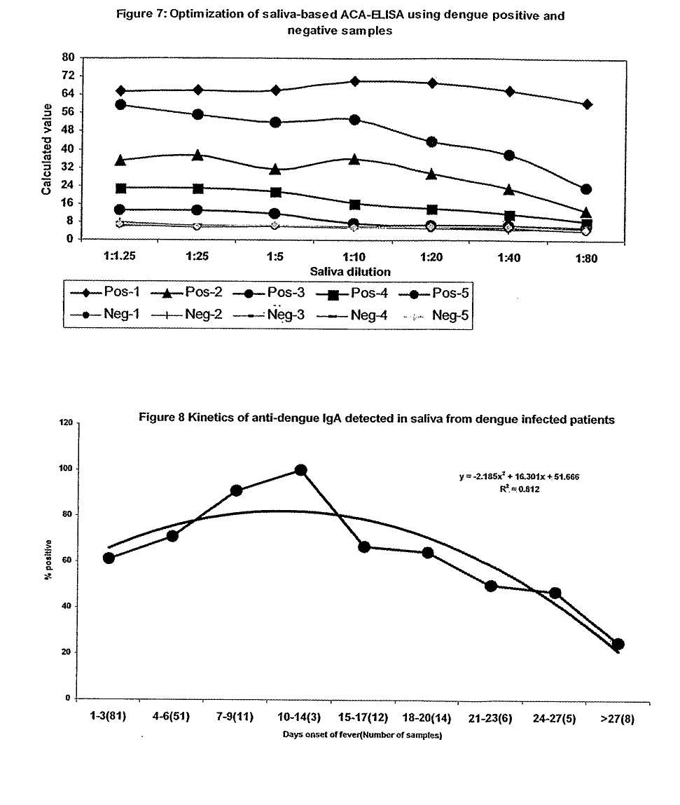

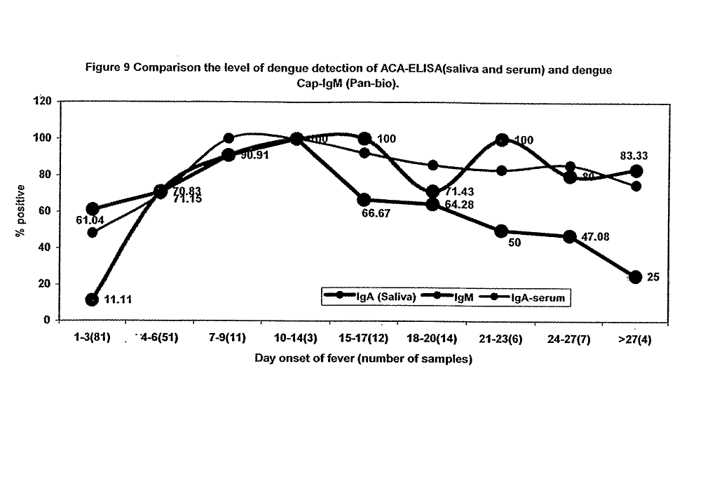

On the other hand, the detection levels of AAC-ELISA were 6.49% during the first collection, 41.67% in the second collection and 72.50% in the third collection. Comparatively the presence of anti-dengue IgM during the same period of illness was as follows: 6.67% in the first collection, 66.67% in the second collection and 79.61% in the third collection (FIG. 6). A similar level of performance of ACA-ELISA was observed when it was used to detect anti-dengue IgA using hospital samples (FIG. 7).

The newly developed ACA-ELISA showed better performance compared to ACC-ELISA. This is because of eliminating the interference of non-dengue specific IgA in ACA-ELISA which inhibits 43.71% to 79.79 % during AAC-ELISA, particularly in early phase of dengue illness. The ACA-ELISA also detected more dengue cases compared to MAC-ELISA, which may be due to the lack of dengue IgM production in secondary infection (Chanama et. al.2004) where IgA was found 92.1% in association with IgG. This study suggests that ACA-ELISA can be used alone or in combination with MAC ELISA to detect more dengue cases (76.26% among dengue confirmed cases).

### Example 6

### Development of ACA-ELISA Using Saliva

a) Antigen capture anti-dengue IgA ELISA (ACA-ELISA): The 96 well plate (Max-absorb-NUNC) was coated with 100 μl per well of pan-dengue MAb diluted at 1:1000 in coating buffer and incubated either over-night at 4° C. or 1 hour at 37° C. After blocking of the plate with blocking buffer for 1 hour at 37° C., dengue lysate antigen was added at 1:100 dilutions to each well and incubated at RT for 1 hour. The plate was then washed 4 times using washing buffer and 100 μl of test saliva at 1:5 in diluent buffer was added to each well. 1 positive and 3 negative serum controls were kept in each plate. After 1 hour of incubation at RT, the plate was washed again 6 times using washing buffer and 100 μl of rabbit anti-human IgA conjugated with HRP (1:4000, Dakocytomation, Denmark) was added to each well and then incubated for 30 minutes at RT. Following incubation, the plate was washed again 6 times and 100 μl of OPD (Sigma, USA) was added to each well and incubated for 5 minutes at RT. The further colour development was stopped using sulphuric acid (2.75%) and the plate read in an ELISA reader at 492 nm. Results were calculated using a formula of OD value of the test sample divided by an average OD of negative samples and multiplying the value by 5.

b) Standardization of assays: Checkerboard titrations showed that 100 μl pan dengue MAb (1:4000 in coating buffer, 0.25 ng/well) and 100 μl of lysate antigen at dilution 1:100 was found as optimum for the antigen in 96 well plates. The optimal dilution of saliva samples used in ELISA was determined by checkerboard titration against anti-human IgA using saliva from 5 dengue-confirmed cases (PCR) and 5 saliva samples from healthy donors as negative controls. Similarly, the optimal dilutions of rabbit anti-human IgA-HRP for ACA-ELISA and anti-mouse IgG for MC-ELISA were determined by checkerboard titration at 1:4000 and 1:3000 respectively.

5 saliva samples were collected from dengue PCR-positive patients on day 5-7 and 5 samples from healthy volunteers were tested at ranges of 1:1:25 to 1:640 in diluent buffer. Results showed that all saliva samples from five dengue-confirmed patients showed high levels of anti-dengue IgA against dengue lysate antigen titres from 1:160 to 1:640 while 5 dengue negative saliva samples did not show any reaction even at lower dilutions (1-2.5) except 2 which showed a mild reaction up to a level of 1:1:25 dilutions (FIG. 6). Hence, the cut-off point of saliva dilution for the ACA-ELISA was set at 1:5 (4 times) and used throughout the study.

The ACA-ELISA was developed using 184 dengue PCR-confirmed saliva samples collected at acute and convalescent stages: day-1-3, day-4-7 and day-10-37. 104 saliva samples were collected from patients who had a fever but were negative to dengue-PCR test. 50 saliva samples collected from healthy patients were also used as negative samples in this study. 100 ul of pan-dengue MAb (1:4000 in coating buffer, 0.25 ng/well) and 100 ul of lysate antigens at 1:100 dilution was found as optimum for the antigen in 96-well plates.

### Example 7

### Detection of Anti-Dengue IgA Using AAC- and ACA-ELISAs

The presence of anti-dengue IgA in saliva and serum samples from 106 dengue confirmed patients were tested using 2 anti-dengue IgA assays (MC-ELISA and ACA-ELISA). Results showed that 63.04% of saliva and 48.08% of serum samples were positive to dengue IgA by ACA-ELISA while 32.40% of saliva and 37.15% of serum were positive to MC-ELISA.

The kinetics of anti-dengue IgA produced in saliva during dengue illness was displayed by ACA-ELISA showing dengue reactive IgA appeared during early phase of infection and reached 100% within the second week of illness (onset of fever) and gradually declined after the fifth week (FIG. 10).

The level of dengue-positive cases detected by ACA-ELISA during 3 serum collections at acute and convalescent stages (day 1-3, day 4-7 and day 10-37 of onset of fever) were 61.04%, 70.83% and 54.35% respectively. Compared to dengue MAC-ELISA, saliva-based ACA-ELISA picked up 50% more dengue cases between day 1-3 (FIG. 11) and this clearly indicated the effectiveness of this technique in the early detection of dengue infection using non-invasive samples such as saliva.

TABLE 3
2-by-2 tables show the sensitivity, specificity, positive and
negative predictive values of saliva-based ACA-ELISA on 184 saliva
samples collected between day 1 to 37 of onset of fever.

| | | Dengue PCR | | |
|---|---|---|---|---|
| | | Positive | Negative | Total |
| Saliva | Positive | 116 | 6 | 122 |
| ACA- | Negative | 68 | 148 | 216 |
| ELISA | Total | 184 | 154 | 338 |
| | Sensitivity (%) | 63.04 | | |

| | |
|---|---|
| Specificity (%) | 96.10 |

Positive predictive value = 91.89%
Negative predictive value = 79.57%

### REFERENCES

Bravo, J. R., M. G. Guzman, and G. P. Kouri. 1987. Why dengue haemorrhagic fever in Cuba? I. Individual risk factors for dengue haemorrhagic fever/dengue shock syndrome (DHF/DSS). Trans. R. Soc. Trop. Med. Hyg. 81:816-820.

Balmaseda, A., M. G. Guzman, S. Hammond, G. Robleto, C. Flores, Y. Tellez, E. Videa, S. Saborio, L. Perez, E. Sandoval, Y. Rodriguez, and E. Harris. 2003. Diagnosis of dengue virus infection by detection of specific immunoglobulin M (IgM) and IgA antibodies in serum and saliva. Clin. Diagn. Lab. Immunol. 10:317-322.

Burke, D. S., A. Nisalak, D. E. Johnson, and R. M. Scott. 1988. A prospective study of dengue infections in Bangkok. Am. J. Trop. Med. Hyg. 38:172-180.

Deubel, V., R. M. Kinney, and D. W. Trent. 1988. Nucleotide sequence and deduced amino acid sequence of the nonstructural proteins of dengue type 2 virus, Jamaica genotype: comparative analysis of the full-length genome. Virology 165:234-244.

Gentry, M. K., E. A. Henchal, J. M. McCown, W. E. Brandt, and J. M. Dalrymple. 1982. Identification of distinct determinants on dengue-2 virus using monoclonal antibodies. Am. J. Trop. Med. Hyg. 31:548-555.

Gibbons, R. V., and D. W. Vaughn. 2002. Dengue: an escalating problem. BMJ 324:1563-1566.

Groen, J., Koraka, P., Velzing, J., Copra, C., Osterhaus, A. D. (2000). Evaluation of six immunoassays for detection of dengue virus-specific immunoglobulin M and G antibodies. Clin Diagn Lab Immunol. 7(6):867-71.

Gubler, D. J., Sather, G. E. (1988). Laboratory diagnosis of dengue and dengue hemorrhagic fever. In A. Homma, and J. F. Cunha (ed.), Proceedings of the International Symposium on Yellow Fever and Dengue. p. 291-322.

Gubler, D. J. 1996. Serologic diagnosis of dengue/dengue haemorrhagic fever. Dengue Bull. 20:20-23. Gubler, D. J. (1998). Dengue and dengue hemorrhagic fever. Clin Microbiol Rev. 11 (3):480-96.

Gubler, D. J. 1998. Dengue and dengue hemorrhagic fever. Clin. Microbiol. Rev. 11:480-496.

Gubler, D. J. (2002). Epidemic dengue/dengue hemorrhagic fever as a public health, social and economic problem in the 21st century. Trends Microbiol. 10(2):100-3. Innis, B. L., A. Nisalak, S. Nimmannitya, S. Kusalerdchariya, V. Chongswasdi, S. Suntayakorn, P. Puttisri, and C. H. Hoke. 1989. An enzyme-linked immunosorbent assay to characterize dengue infections where dengue and Japanese encephalitis co-circulate. Am. J. Trop. Med. Hyg. 40:418-427.

Halstead, S. B., H. Shotwell, and J. Casals. 1973. Studies on the pathogenesis of dengue infection in monkeys. II. Clinical laboratory responses to heterologous infection. J. Infect. Dis. 128:15-22.

Halstead, S. B. 1988. Pathogenesis of dengue: challenge to molecular biology. Science 239:476-481.

Henchal, E. A., J. M. McCown, M. C. Seguin, M. K. Gentry, and W. E. Brandt. 1983. Rapid identification of dengue virus isolates by using monoclonal antibodies in an indirect immunofluorescence assay. Am. J. Trop. Med. Hyg. 32:164-169.

Leyssen, P., E. D. Clercq, and J. Neyts. 2000. Perspectives for the treatment of infections with Flaviviridae. Clin. Microbiol. Rev. 13:67-82.

Nawa, M., Takasaki, T., Yamada, K.l., Akatsuka, T., Kurane, I. (2001). Development of dengue IgM-capture enzyme-linked immunosorbent assay with higher sensitivity using monoclonal detection antibody. J Virol Methods. 92(1):65-70.

Koraka, P., C. P. Burghoorn-Maas, A. Falconar, T. E. Setiati, K. Djamiatun, J. Groen, and A. D. M. E. Osterhaus. 2003. Detection of immune-complex-dissociated nonstructural-1 antigen in patients with acute dengue virus infections. J. Clin. Microbiol. 41:4154-4159.

Oliveira, D. P. S., L. D. Malta, M. Clotteau, N. R. R. J. Pires, and F. B. A. Lopes. 2003. Improved detection of dengue-1 virus from IgM-positive serum samples using C6/36 cell cultures in association with RT-PCR. Intervirology 46:227-231.

Ooi, E. E., Hart, T. J., Tan, H. C., Chan, S. H. (2001). Dengue seroepidemiology in Singapore. Lancet. 357(9257):685-6.

Platt, K. B., Linthicum, K. J., Myint, K. S., Innis, B. L., Lerdthusnee, K., Vaughn, D. W. (1997). Impact of dengue virus infection on feeding behavior of Aedes aegypti. Am J Trop Med Hyg. 57(2):119-25.

Scott, T. W., Naksathit, A., Day, J. F., Kittayapong, P., Edman, J. D. (1997). A fitness advantage for Aedes aegypti and the viruses it transmits when females feed only on human blood. Am J Trop Med Hyg. 57(2):235-9.

Shu, P. Y., L. K. Chen, S. F. Chang, Y. Y. Yueh, L. Chow, L. J. Chien, C. Chin, T. H. Lin, and J. H. Huang. 2003. Comparison of capture immunoglobulin M (IgM) and IgG enzyme-linked immunosorbent assay (ELISA) and nonstructural protein NS1 serotype-specific IgG ELISA for differentiation of primary and secondary dengue virus infections. Clin. Diagn. Lab. Immunol. 10:622-630.

Simmons, M., Jennings, G., Oprandy, J. J. (1993). A rapid membrane based immunobinding assay for the detection of dengue virus in tissue culture. Southeast Asian J Trop Med Public Health. 24(4):742-6.

Stollar, V., Stevens, T. M., Schlesinger, R. W. (1966). Studies on the nature of dengue viruses. II. Characterization of viral RNA and effects of inhibitors of RNA synthesis. Virology. 30(2):303-12.Newton, E. A., Reiter, P. (1992). A model of the transmission of dengue fever with an evaluation of the impact of ultra-low volume (ULV) insecticide applications on dengue epidemics. Am J Trop Med Hyg. 47(6):709-20.

Talarmin, A., B. Labeau, J. Lelarge, and J. L. Sarthou. 1998. Immunoglobulin A-specific capture enzyme-linked immunosorbent assay for diagnosis of dengue fever. J. Clin. Microbiol. 36:1189-1192.

World Health Organization. 1997. Dengue haemorrhagic fever: diagnosis, treatment, prevention and control, 2nd ed. World Health Organization, Geneva, Switzerland.

World Health Organization. 1997. Dengue haemorrhagic fever: diagnosis, treatment, prevention and control, 2nd ed. World Health Organization, Geneva, Switzerland.

World Health Organization. 1999. Strengthening implementation of the global strategy for dengue fever and dengue haemorrhagic fever, prevention and control. Report of the informal consultation, 18-20 October. World Health Organization, Geneva, Switzerland.

World Health Organization: Fact sheet No: 117. (2002). Dengue and Dengue Haemorrhagic fever. http://www.who.int/inf-fs/en/fact117.html

Wu, S. J., Paxton, H., Hanson, B., Kung, C. G., Chen, T. B., Rossi, C., Vaughn, D. W., Murphy, G. S., Hayes, C. G. (2000). Comparison of two rapid diagnostic assays for detection of immunoglobulin M antibodies to dengue virus. Clin Diagn Lab Immunol. 7(1):106-10.

Finally it is to be understood that various other modifications and/or alterations may be made without departing from the spirit of the present invention as outlined herein.

1. A method for detecting IgA in a subject that is specific for a flavivirus, said method comprising:
contacting a biological sample from the subject with a mixture of flavivirus specific immunogenic components captured from a lysate of cells infected with flavivirus;
determining the presence of a complex that forms between a binding partner in the biological sample and the flavivirus specific immunogenic components; and
characterizing the binding partner in the complex with an anti-IgA antibody.

2. A method for detecting exposure of a subject to a IgA specific flavivirus, said method comprising:
contacting a biological sample from the subject with a mixture of flavivirus specific immunogenic components captured from a lysate of cells infected with flavivirus;
determining the presence of a complex that forms between a binding partner in the biological sample and the flavivirus specific immunogenic components;
characterizing the binding partner in the complex; and
correlating the binding partner to exposure to the flavivirus.

3. A method according to claim 1 wherein the flavivirus specific immunogenic components are captured by a monoclonal antibody.

4. A method according to claim 1 wherein the flavivirus specific immunogenic component is selected from the group consisting of flavivirus structural and non-structural proteins, flavivirus particles and fragments thereof, glycoproteins, lipids and carbohydrates derived from the flavivirus.

5. A method according to claim 4 wherein the structural protein is selected from the group consisting of envelope proteins, Pr membrane proteins, and nucleocapsid proteins.

6. A method according to claim 5 wherein the structural protein is an envelope protein.

7. A method according to claim 1 wherein the flavivirus specific immunogenic component is selected from the group consisting of dengue virus serotype immunogenic components selected from the group including DEN-1, DEN-2, DEN-3 and DEN-4.

8. A method according to claim 2 wherein the method detects exposure to a dengue virus serotype selected from the group consisting of DEN-1, DEN-2, DEN-3 and DEN-4,

9. A method according to claim 4 wherein the non-structural protein is selected from the group consisting of including NS-1, NS-2a, NS-2b, NS-3, NS-4a, NS-4b and NS-5.

10. A method according to claim 9 wherein the non-structural protein is NS-1,

11. A method according to claim 1 wherein the flavivirus specific immunogenic component is an anti-idiotypic antibody to an antigen binding site of a flavivirus antibody generated in response to exposure to a component derived from flavivirus.

12. A method according to claim 1 wherein the binding partner is a flavivirus specific antibody or an immunological fragment thereof.

13. A method according to claim 12 wherein the binding partner is an antibody expressed in an early stage of a flavivirus infection, during convalescence or derived from a previous infection.

14. A method according to claim 1 wherein the binding partner is an IgA antibody.

15. A method according to claim 1 wherein the flavivirus is selected from the group consisting of yellow fever virus, dengue virus, and JE virus.

16. A method according to claim 15 wherein the flavivirus is dengue virus.

17. A method according to claim 12 wherein the binding partner antibody is an IgA antibody that is specific to a dengue serotype selected from the group consisting of DEN-1, DEN-2, DEN-3 and DEN-4.

18. A method according to claim 1 wherein the biological sample is selected from the group consisting of blood, saliva, cord fluid, B cells, T cells, plasma, serum, urine and amniotic fluid.

19. A method according to claim 18 wherein the biological sample is serum or saliva.

20. A method according to claim 2 wherein the binding partner is characterized using an anti-IgA antibody.

21. A method according to claim 1 wherein the anti-IgA antibody is bound to a reporter group.

22. A method according to claim 21 wherein the reporter group is an enzyme.

23. A solid support for use in a method according to claim 1, wherein said support comprises flavivirus specific immunogenic components immobilized on the support

24. A solid support according to claim 23, selected from the group consisting of a bead, a disc, a magnetic particles OF a fiber optic sensor, a microtitre plate, a glass slide, a biological microchip and a membrane selected from the group consisting of nitrocellulose, polytetrafluorethylene, cellulose acetate and cellulose nitrate with a filter paper carrier.

25. A kit for detecting IgA in a subject that is specific for a flavivirus or for detecting flavivirus exposure comprising:
a solid support comprising a flavivirus specific immunogenic component captured from a lysate of cells infected with flavivirus; or
a solid support comprising a flavivirus specific immunogenic component captured from a lysate of cells infected with flavivirus attached to a second support;
at least one detection agent conjugated to a reporter group for detecting a binding partner in a biological sample that forms a complex with the flavivirus specific immunogenic component; and optionally
instructions for using said kit to further identify the binding partner of the complex.

26. A kit according to claim 25 wherein the flavivirus specific immunogenic component is immobilized on a solid support.

27. A kit according to claim 25 wherein the flavivirus specific immunological agent is captured by a monoclonal antibody.

28. A kit according to claim 27 wherein the flavivirus specific immunogenic component is selected from the group consisting of flavivirus virus structural and non-structural proteins, flavivirus particles or fragments thereof, glycoproteins, lipids and carbohydrates derived from the flavivirus.

29. A kit according to claim 28 wherein the structural protein is selected from the group consisting of envelope proteins Pr membrane proteins, and nucleocapsid proteins.

30. A kit according to claim 29 wherein the structural protein is an envelope protein.

31. A kit according to claim 25 wherein the flavivirus is selected from the group consisting of yellow fever virus, dengue virus, and JE virus.

32. A kit according to claim 31 wherein the flavivirus is dengue virus.

33. A kit according to claim 28 wherein the flavivirus specific immunogenic component is selected from the group consisting of dengue virus serotype immunogenic components DEN-1, DEN-2, DEN-3 and DEN-4.

34. A kit according to claim 28 wherein the non-structural protein is selected from a group consisting of NS-1, NS-2a, NS-2b, NS-3, NS-4a, NS-4b and NS-5.

35. A kit according to claim 34 wherein the non-structural protein is NS-1.

36. A method of assessing the relative risk of one or more subjects being exposed to a IgA specific flavivirus within a defined location comprising:
obtaining samples from a representative population within a defined location;
assessing evidence of exposure of individual members of a sample population to a flavivirus by the method comprising the steps of
contacting a biological sample from the subject with a mixture of flavivirus specific immunogenic components captured from a lysate of cells infected with flavivirus; and
determining the presence of a complex that forms between a binding partner in the biological sample and the flavivirus specific immunogenic component and wherein the presence of the complex is indicative of exposure of the subject to a flavivirus; and
assessing the relative risk of exposure within the defined location by characterizing the binding partner in the complex.

37. The method of claim 36, wherein said defined location is a geographical area, a housing estate, a means of transport, or a center for medical treatment or assessment.
